(19) United States
(12) Patent Application Publication
(10) Pub. No.: US 2003/0182733 A1
(43) Pub. Date: Oct. 2, 2003

(54) Use of organic substances

(76) Inventors: Jurgen Geiwiz, Lorrach-Hauningen (DE); Reinhard Pedrazzi, Allschwil (CH); Torsten Henning, Bad Soden Taunus (DE); Friedrich Lehr, Efringen-Kirchen (DE); Georg Schoefberger, Basel (CH)

Correspondence Address: CLARIANT CORPORATION, INTELLECTUAL PROPERTY DEPARTMENT, 4000 MONROE ROAD, CHARLOTTE, NC, 28205, US

(21) Appl. No.: 10/296,274

(22) PCT Filed: May 22, 2001
(86) PCT No.: PCT/IB01/00887

(30) Foreign Application Priority Data
May 24, 2000 (CH) 1039/00

(51) Int. Cl. A61K 7/13

(52) U.S. Cl. 8/405; 8/406; 8/408; 8/415; 8/421

(57) ABSTRACT

The present invention relates to the use of certain compounds, which are given in claim 1, or mixtures thereof for the coloring and tinting of keratin fibers, in particular of human hair.

The present invention relates to the use of certain compounds or mixtures thereof for the coloring and tinting of keratin fibers, in particular of human hair.

Two coloring processes are generally used for the coloring and tinting of keratin fibers, in particular of human hair. In the first process, the coloration is generated using so-called oxidative or permanent coloring agents or using a mixture of different developer substances and coupler substances and an oxidizing agent. As required, so-called direct (non-oxidative) dyes can be added to round off the coloring or tinting result or to produce particular color effects. The second process uses exclusively direct dyes which are applied to the hair in a suitable carrier mass. This process is simple to use, exceptionally mild and is distinguished by little damage to the keratin fibers. The direct dyes used here are subject to a large number of requirements. For example, the dyes have to be safe from a toxicological viewpoint. The dyes which are used here according to the invention are known as textile and paper dyes and satisfy this criterion.

The present invention provides for the use of compounds of the formula (I)

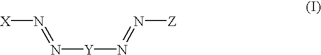

in which X is a radical of the formula X₁

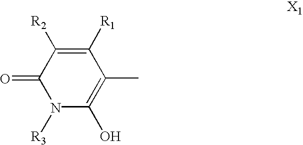

in which

R₁ is hydrogen or a C₁₋₆-alkyl radical, in particular -CH₃ and -CH₂CH₃,

R₂ is hydrogen, cyano, carboxamide or

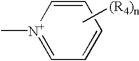

in which R₄ is hydrogen, C₁₋₄-alkyl, in particular -CH₃, -COOH, or -OC₁₋₄alkyl and n is 1 or 2,

R₃ is hydrogen or optionally substituted C ₁₋₆-alkyl, C, ₁₋₆-alkylene-NR₅R₆, where R₅, R₆, independently of one another are hydrogen or C₁₋₄-alkyl,

or in which X is a radical of the formula X₂

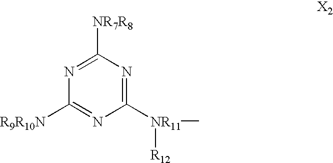

in which R₇, R₈, R₉ and R₁₀, independently of one another, are hydrogen, a C₁₋₆-alkyl, C₁₋₆-alkylene-NR₁₃R₁₄ or

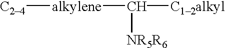

in which R₁₃ and R₁₄, independently of one another are hydrogen or C₁₋₄-alkyl, and R₅ and R₆ have the meanings given above and

in which R₁₁ is hydrogen or C₁₋₄-alkyl and

in which R₁₂ is an optionally substituted phenylene or naphthylene group,

or in which X is an optionally substituted phenyl or naphthyl group,

in which Y is a divalent group of the formula Y₁ or Y₂

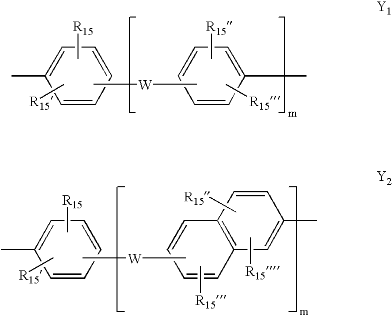

in which R₁₅, R₁₅′, R₁₅″, R₁₅′″and R₁₅″″, independently of one another, are a C₁₋₄-alkyl group, hydroxyl group, a C₁₋₄-alkoxy group and

m is 0 or 1 and

W is a divalent group, in particular C₁₋₆-alkylene, preferably C₁₋₄-alkylene, -N=N-,

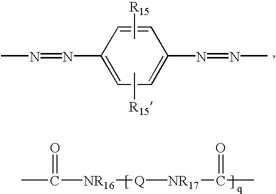

in which R₁₅ and R₁₅′ may have the meanings given above, q can assume the value 0 or 1, Q is C₁₋₄-alkylene and R₁₆ and R₁₇, independently of one another, are hydrogen or a C₁₋₄-alkyl group

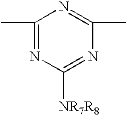

or W is

in which R₇ and R₈ have the meanings given above

and in which Z, independently of X can have the meanings given for X

and/or optionally compounds of the formula (II)

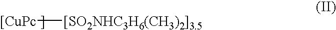

in which Pc is a phthalocyanine radical,

and/or optionally compounds of the formula (III)

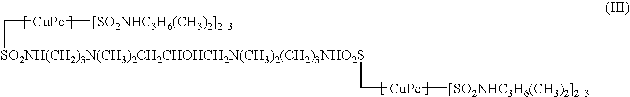

in which Pc is a phthalocyanine radical

and/or optionally iron complexes of the compounds of the formula (IV)

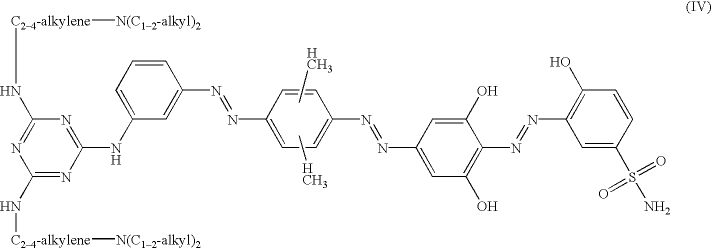

for the coloring or tinting of keratin fibers, in particular of human hair.

It is to be noted that all compounds can also be used in the respective salt form and that all alkyl and alkylene radicals may either be branched or linear.

Particular preference is given to the use of the following compounds according to formula (I) in which X are radicals of the formula X₃-X₇

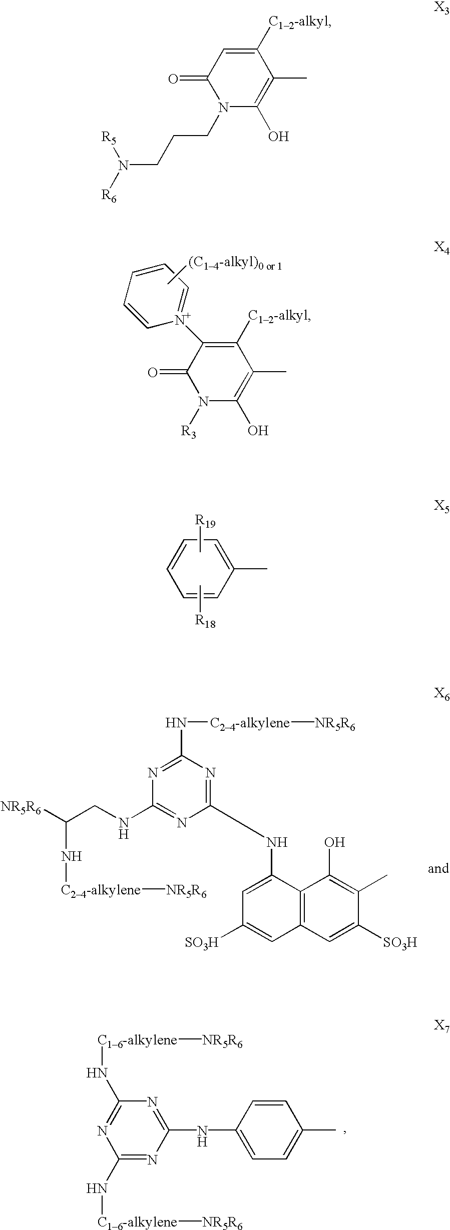

in which R,₁₈ and R₁₉, independently of one another are hydrogen, C ₁₋₄-alkyl, —OC₁₋₄-alkyl, -SO₂NH₂ or hydroxyl, and

R₃, R₅ and R₆ have the meanings given above.

Likewise preferred is the use of compounds according to formula (I), where in Y₁ and Y₂ m is 0 and R₁₅ and R₁₅′ are hydrogen.

Likewise preferred is the use of compounds according to formula (I), where in Y₁ m is 1 and W is W₁ to W₇

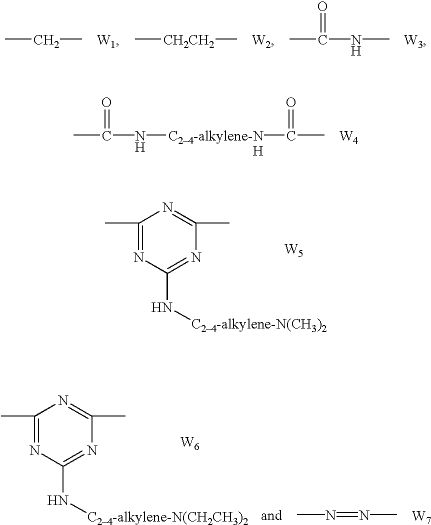

Likewise preferred is the use of compounds according to formula (I) where in Y₂ m is 1 and W is W₇

—N═N—W₇

Likewise preferred is the use of compounds according to formula (I) where Z, independently of X, is X₃-X₇.

Particularly preferred compounds according to formula (I) have the following structures (V)-(XII)

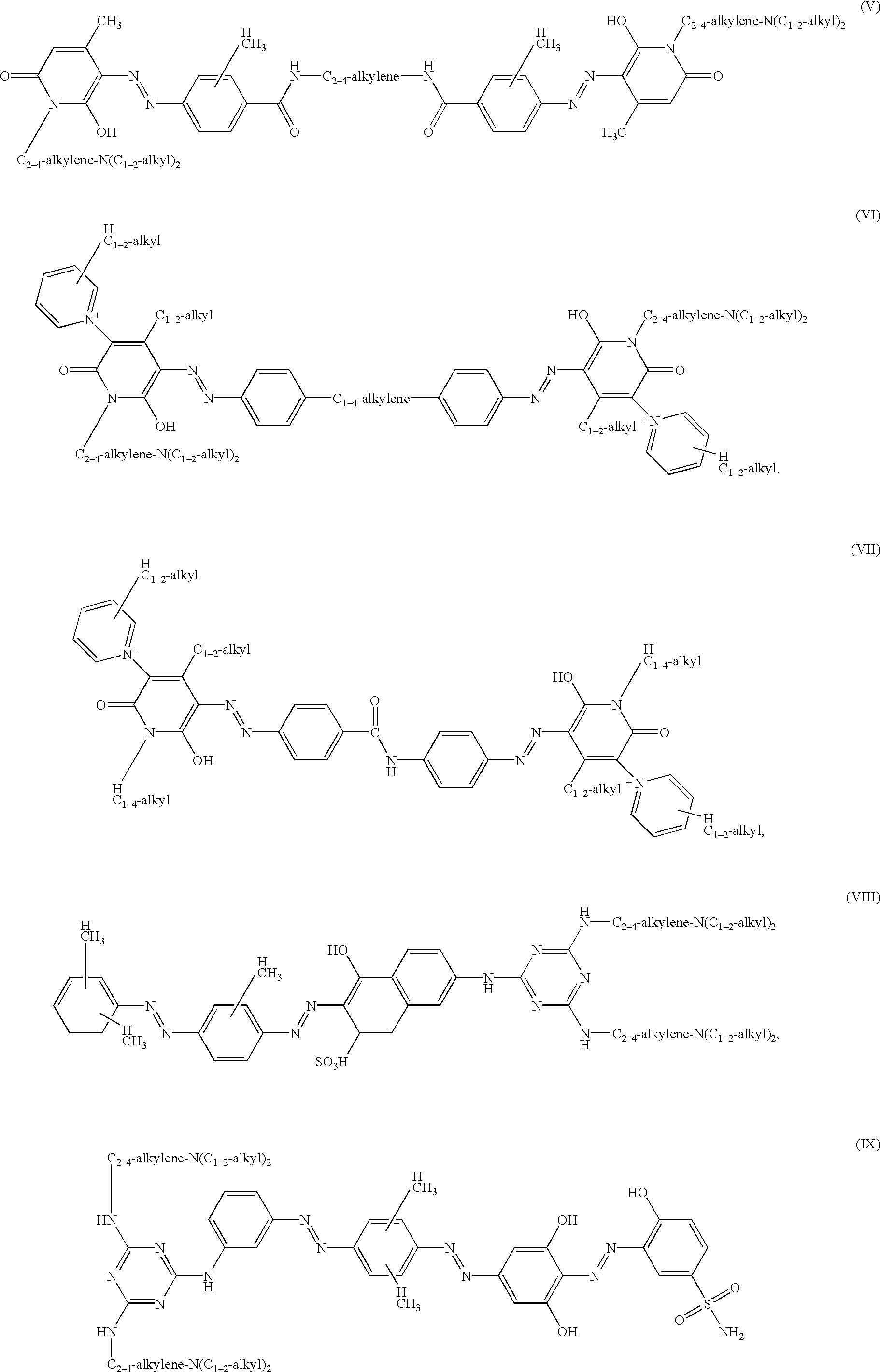

in the form of the iron complex,

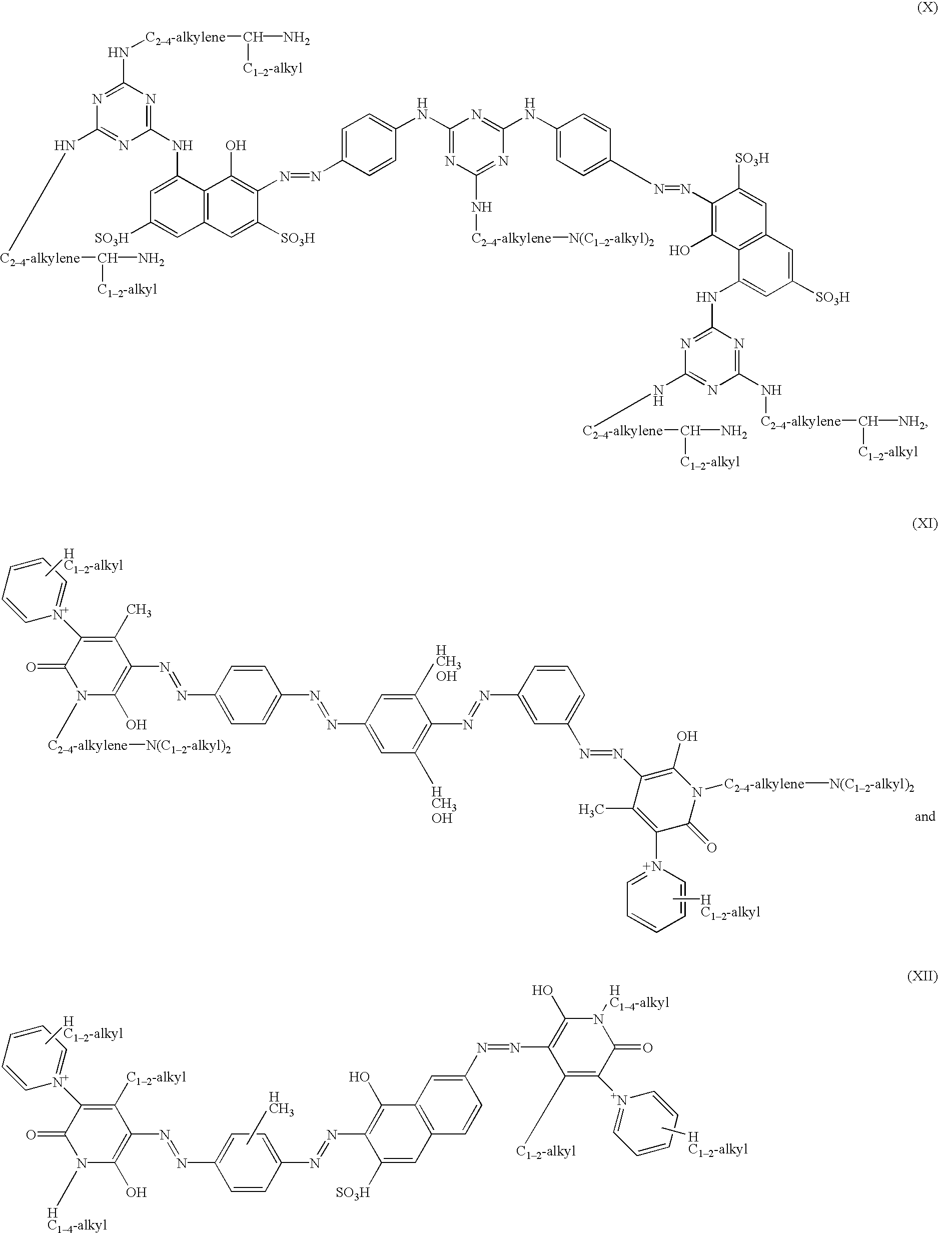

The syntheses for the various compounds given above are known from: U.S. Pat. Nos. 5,352,334, 5,023,324, 5,929,215, 4,367,172, EP 853104 A2, U.S. Pat. No. 5,498,701, 5,084,068 4,665,162 5,352,334, 5,001,226 4,780,106, 4,673,735 and DE 3416117 C2.

Keratin fibers are to be understood as meaning wool, furs, feathers and, in particular, human hair.

For the use according to the invention of the above dyes, they are applied in an aqueous cosmetically compatible medium. The typical pH of such a composition is between 5 and 9, preferably between 6 and 8. To regulate the pH, use is made of generally known, mild inorganic or organic bases, salts of weak acids or buffers. Typical representatives of such compounds are ammonia, ammonium carbonate, potassium carbonate or sodium carbonate, sodium hydroxide and mono-, di-or triethanolamine, disodium hydrogenphosphate, sodium citrate or sodium borate.

The proportion of dyes in the composition is between 0.01 and 10% by weight.

Various auxiliaries, such as surface-active compounds, solubilising agents, thickeners, gel-forming substances, antioxidants, penetration agents, sequestering agents, buffers, perfume oil, light protection agents, care agents, natural dyes, preservatives and hair-cleansing preparations may additionally be present in the composition according to the invention.

Suitable surface-active substances or mixtures thereof are all surface-active substances suitable for use on the human body, where, in principle, anionic and also zwitterionic, ampholytic nonionic and cationic surfactants are suitable. Preference is, however, given in most cases to anionic, zwitterionic or nonionic surfactants.

Examples of anionic surfactants are soaps (linear fatty acids having 10-22 carbon atoms); ether carboxylic acids of the formula R—O—(CH₂CH₂O)ₓ—CH₂—COOH, in which R is a linear alkyl group having 10 to 22 carbon atoms and X ranges from 0 to 16; C₁₀₋₁₈-acyl sarcosides; C₁₀₋₁₈-acyl taurides; C₁₀₋₁₈-acyl isethionates; sulphosuccinic mono C₈₋₁₈-alkyl polyoxyethyl esters having 1 to 6 oxyethyl groups; sulphosuccinic mono-and-di-C₈₋₁₈-alkyl esters; linear C₁₂₋₁₈-alkanesulphonates; alpha-sulpho C₁₂₋₁₈ fatty acid methyl esters; C₁₀₋₁₈-alkyl sulphates; C₁₀₋₁₈-alkyl polyglycol ethersulphates of the formula R—O—(CH₂CH₂O)ₓ—SO₃H, in which R is a preferably linear Cl₁₀₋₁₈-alkyl group and X is 0 to 12; esters of tartaric acid and citric acid with alcohols, which represent addition products of approximately 2-15 molecules of ethylene oxide and/or propylene oxide to fatty alcohols having 8 to 22 carbon atoms.

Examples of zwitterionic surfactants are betaines, such as N-C₈₋₁₈-alkyl-N,N-di-methylammonium glycinates; N-C₈₋₁₈-acylaminopropyl-N,N-dimethylammonium gly-cinates; 2-C₈₋₁₈-alkyl-3-carboxymethyl-3-hydroxyethylimidazolines and cocoacylamino-ethyl hydroxyethylcarboxymethyl glycinate.

Examples of nonionic surfactants are addition products of from 2 to 30 mol of ethylene oxide and/or 0 to 5 mol of propylene oxide to linear fatty alcohols having 8 to 22 carbon atoms and to alkylphenols having 8 to 15 carbon atoms in the alkyl group; C₈₋₁₈ fatty acid mono- and diesters of addition products of from 1 to 30 mol of ethylene oxide to glycerol; C₈₋₂₂-alkyl mono- and oligoglycosides and ethoxylated analogues thereof; addition products of from 5 to 60 mol of ethylene oxide to castor oil and hydrogenated castor oil; addition products of ethylene oxide to sorbitan fatty acid esters; addition products of ethylene oxide to fatty acid alkanolamides. The proportion of such surface-active substances in the composition is between 0.5 and 40% by weight.

Suitable solubilising agents are C₁₋₄-alcohols, glycols such as ethylene glycol and propylene glycol, 2-methoxyethanol, 2-ethoxyethanol or 2-butoxyethanol. The proportion of such solubilising agents in the composition is between 0 and 40% by weight.

Suitable thickeners are cellulose derivatives, sodium alginates, gum arabic, xanthan gum, tragacanth, acrylic acid polymers, polyvinylpyrrolidone, vinyl acetate-crotonic acid copolymers, vinyl acetate-vinylpyrrolidone copolymers, butyl vinyl ether-maleic anhydride copolymers or inorganic thickeners such as bentonites. The proportion of thickeners in the composition is between 0 and 5% by weight.

Suitable gel-forming substances are carbomers. The proportion of gel-forming substances in the composition is between 0 and 5% by weight.

Suitable care agents are panthenol or lanolin derivatives. The proportion of care agents in the composition is between 0 and 5% by weight.

Suitable natural dyes are, in particular henna red, henna neutral, henna black, camomile blossom, sandalwood, buckthorn bark, sage, logwood, madder root, catechu, sedre and alkanna root.

For the preparation of compositions for the use according to the invention, further solvents, such as, for example, aliphatic alcohols, in particular ethanol or isopropanol, or glycol ethers, in particular 1,2-propanediol, can be used in addition to water, where the water content in relation to the other solvents is generally about 20 to 100% by weight, while the content of the other solvents is about 0 to 80% by weight.

The dyes can be used according to the invention in liquid form, but also in thickened form as cream, emulsion, paste or gel, or in another form practicable for the purpose.

As coloring composition for the composition according to the invention for the tinting and coloring of keratin fibers, preference is given to the following examples.

Formulation example for a coloring shampoo:

| | |
|---|---|
| 0.5 g | of a compound of the formula (I) and/or of the formula (II) |
| | and/or of the formula (III) and/or of the formula (IV) |
| 0.5 g | of ethoxylated castor oil, 40 mol of ethylene oxide in the |
| | molecule (Cremophor ® EL from BASF) |
| 0.5 g | of perfume |
| 60 g | of isopropanol |
| 38.5 g | of water |

Formulation example for a coloring paste:

| | |
|---|---|
| 0.2 g | of a compound of the formula (I) and/or of the formula (II) and/or |
| | of the formula (III) and/or of the formula (IV) |
| 7 g | of titanium oxide |
| 15 g | of Aerosil ® (manufacturer: Degussa) |
| 10 g | of Lutrol ® (polyethylene glycol from BASF) |
| 66 g | of water |

Preferably, preservatives are also added.

For the use according to the invention of the above dyes, the above compositions can be used in undiluted form or in a 1:1 to 1:10 dilution with water, depending on the desired color intensity.

The coloring time for keratin fibers with the above dyes is between 5 and 50 minutes, preferably between 10 and 30 minutes. The dye composition is left to act for the appropriate time and then the keratin fibers are thoroughly rinsed.

If heat is simultaneously applied during the contact phase, the coloring time can be decisively shortened, or the coloration can be intensified in this way for the same coloring time. The preferred temperatures are between 20 and 40° C.

### FORMULATION EXAMPLE 1

0.5 g of a compound of the formula 1 according to formula (I)

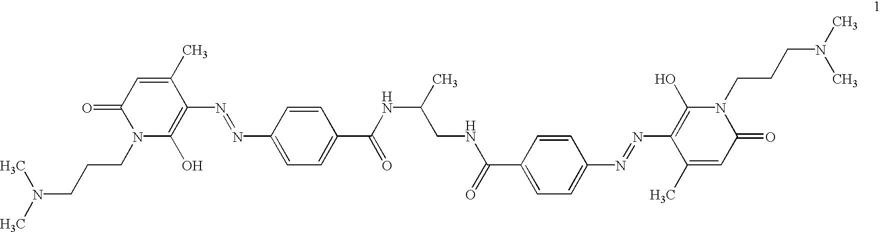

5 g of ethoxylated castor oil, 40 mol of ethylene oxide in the molecule (Cremophor® EL from BASF)

0.5 g of perfume

60 g of isopropanol

38.5 g of water

Analogously to Formulation Example 1, the compounds according to the formulae (V)-(XII) in particular can be used as dyes.

What is claimed is:

1. Use of compounds of the formula (I) 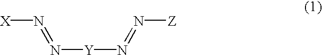
in which X is a radical of the formula X₁ 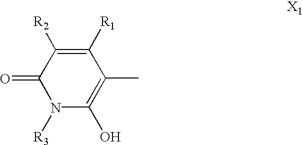
in which
R₁ is hydrogen or a C₁₋₆-alkyl radical,
R₂ is hydrogen, cyano, carboxamide or 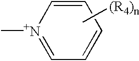
in which R₄ is hydrogen, C₁₋₄, —alkyl, —COOH, or—OC₁₋₄alkyl and
n is 1 or 2,
in which R₃ is hydrogen or optionally substituted C₁₋₆-alkyl, C₁₋₆-alkylene-NR₅R₆,
where R₅, R₆, independently of one another are hydrogen or C₁₋₄-alkyl,
or in which X is a radical of the formula X₂ 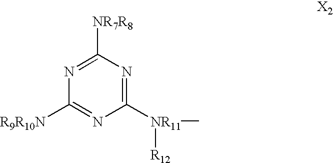
in which R₇, R₈, R₉ and R₁₀, independently of one another, are hydrogen, a C₁₋₆-alkyl, C₁₋₆-alkylene-NR₁₃R₁₄or 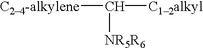
in which R₁₃ and R₁₄, independently of one another are hydrogen or C₁₋₄-alkyl, and R₅ and R₆ have the meanings given above, and
in which R₁₁ is hydrogen or C₁₋₄-alkyl and in which R₁₂ is an optionally substituted phenylene or naphthylene group,
or in which X is a phenyl group of formula X₅ or an optionally substituted naphthyl group. 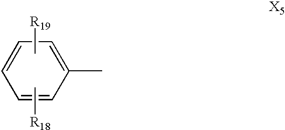
in which R₁₈ and R₁₉, independently of one another are hydrogen, C₁₋₄-alkyl, —OC₁₋₄-alkyl, —SO₂NH₂ or hydroxyl,
in which Y is a divalent group of the formula Y₁ or Y₂ 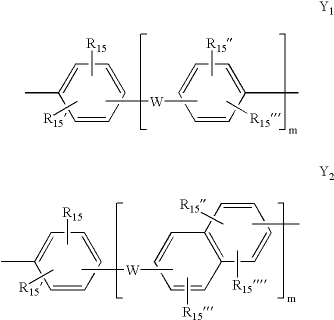
in which R₁₅, R₁₅′, R₁₅″, R₁₅′″R₁₅″″independently of one another, are a C₁₋₄-alkyl group, hydroxyl group, a C₁₋₄-alkoxy group and
m is 0 or 1 and
W is a divalent group,
and in which Z, independently of X, can have the meanings given for X
and/or optionally compounds of the formula (II) 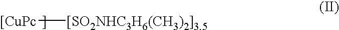
in which Pc is a phthalocyanine radical,
and/or optionally compounds of the formula (III) 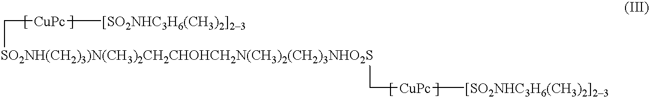
in which Pc is a phthalocyanine radical
and/or optionally iron complexes of the compounds of the formula (IV) 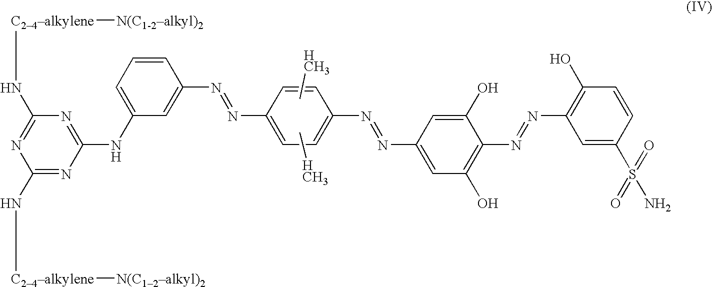
for the coloring or tinting of keratin fibers.

2. Use according to claim 1 for the coloring and tinting of human hair.

3. Use according to claim 1 or 2, characterized in that R₁ and R₄ are —CH₃, and W is C₁₋₆-alkylene, —N═N—, 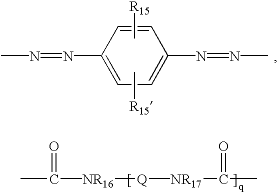
in which R₁₅ and R₁₅′ may have the meanings given in claim 1, q can assume the value 0 or 1, Q is C₁₋₄-alkylene, and R₁₆ and R₁₇, independently of one another, are hydrogen or a C₁₋₄-alkyl group,
or W is 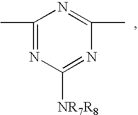
in which R₇ and R₈ have the meanings given in claim 1.

4. Use according to any one of claims 1 to 3, characterized in that X and Z, independently of one another, are one of the radicals X₃ - X₇ 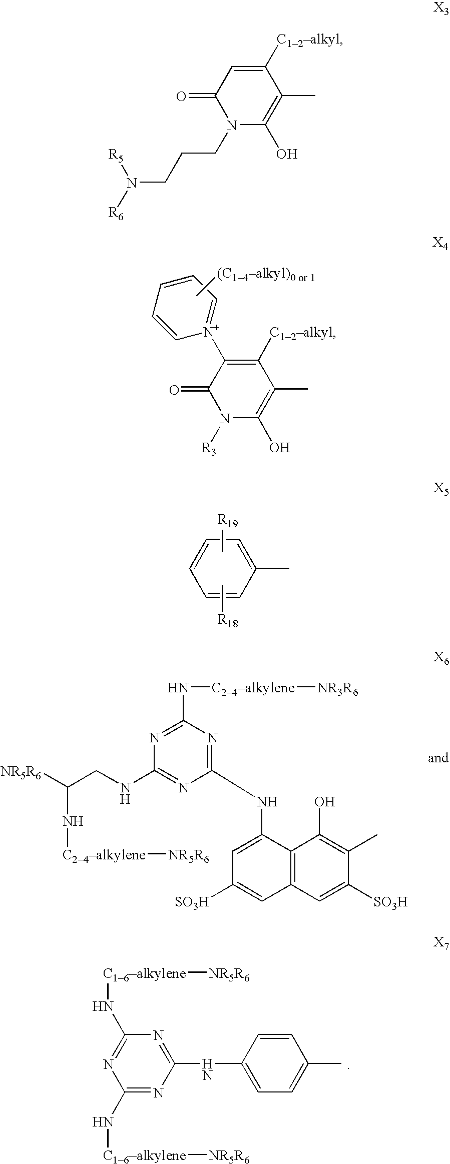
in which R₁₈ and R₁₉, independently of one another are hydrogen, C₁₋₄-alkyl, —OC₁₋₄-alkyl, —SO₂NH₂ or hydroxyl and
R₃, R₅ and R₆ have the meanings given in claim 1, and in
Y₁ and Y₂, m is 0 and R₁₅ and R₁₅′ are hydrogen.

5. Use according to any one of claims 1 to 3, characterized in that X and Z, independently of one another, are one of the radicals X₃-X₇ 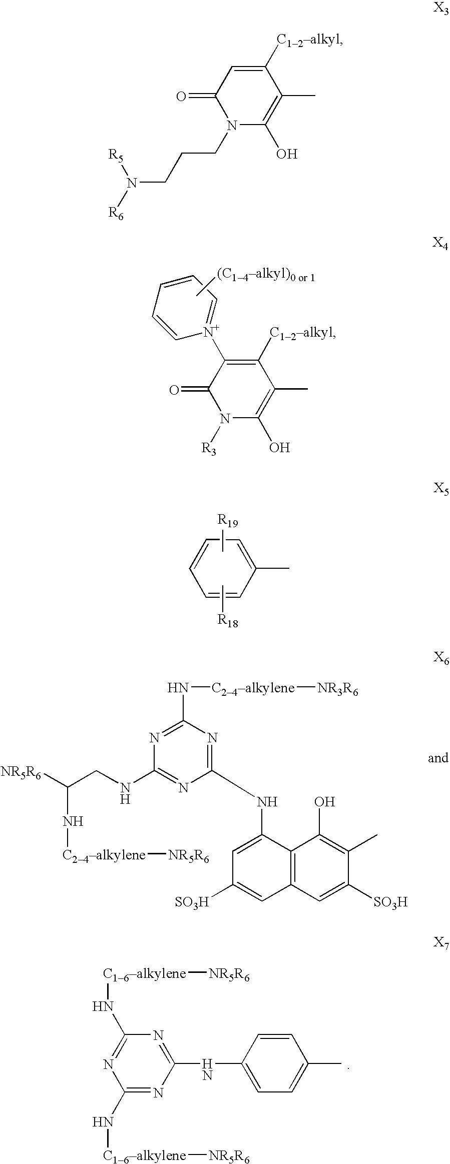
in which R₁₆ and R₁₉, independently of one another are hydrogen, C₁₋₄-alkyl, —OC₁₋₄-alkyl, —SO₂NH₂ or hydroxyl and
R₃, R₅ and R₆ have the meanings given in claim 1, and
Y is Y₁, in which m is 1 and W is W₁ to W₇

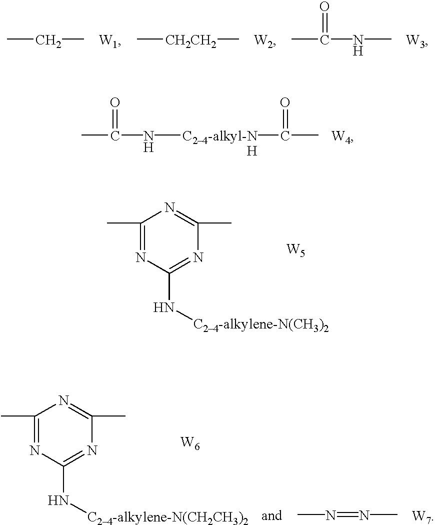

6. Use according to any one of claims 1 to 3, characterized in that X and Z, independently of one another, are one of the radicals X₃ -X₇ 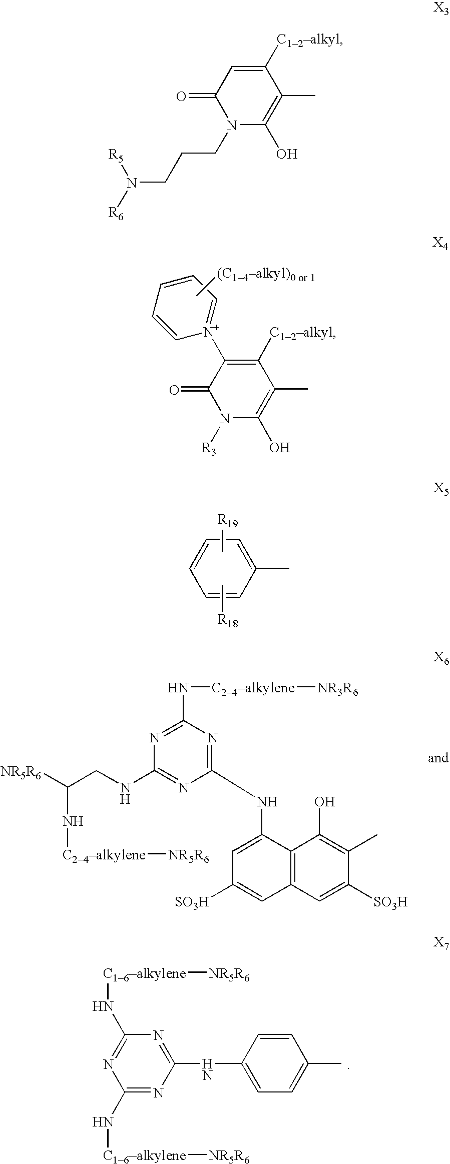
in which R₁₈ and R₁₉, independently of one another are hydrogen, C₁₋₄-alkyl, —OC₁₋₄-alkyl, —SO₂NH₂ or hydroxyl and
R₃, R₅ and R₆ have the meanings given above, and
Y is Y₂, in which m is 1 and W is W₇
—N═N—W₇

7. Use according to claim 1, characterized in that the following compounds having the following structures (V)-(XII) are used

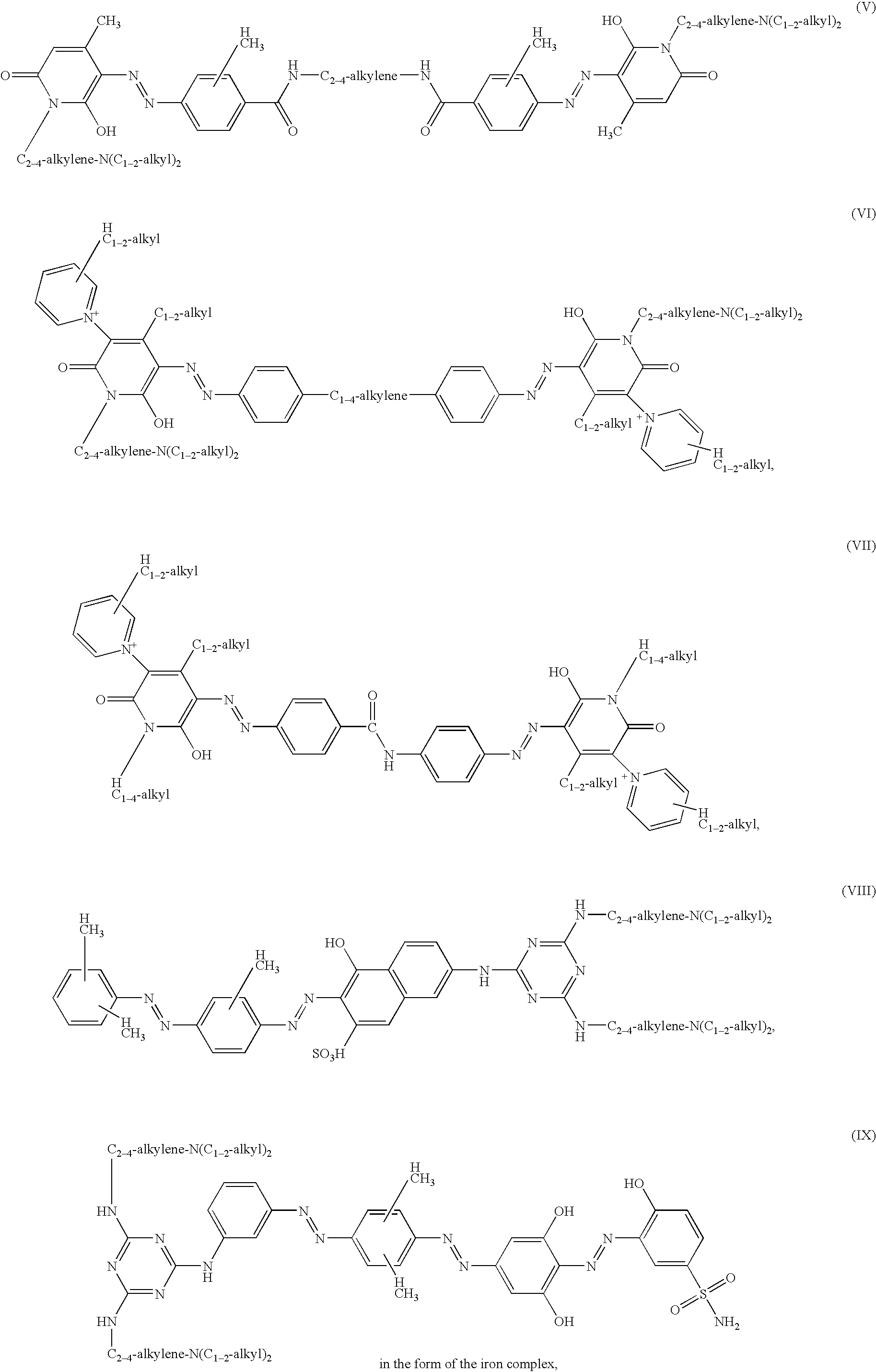

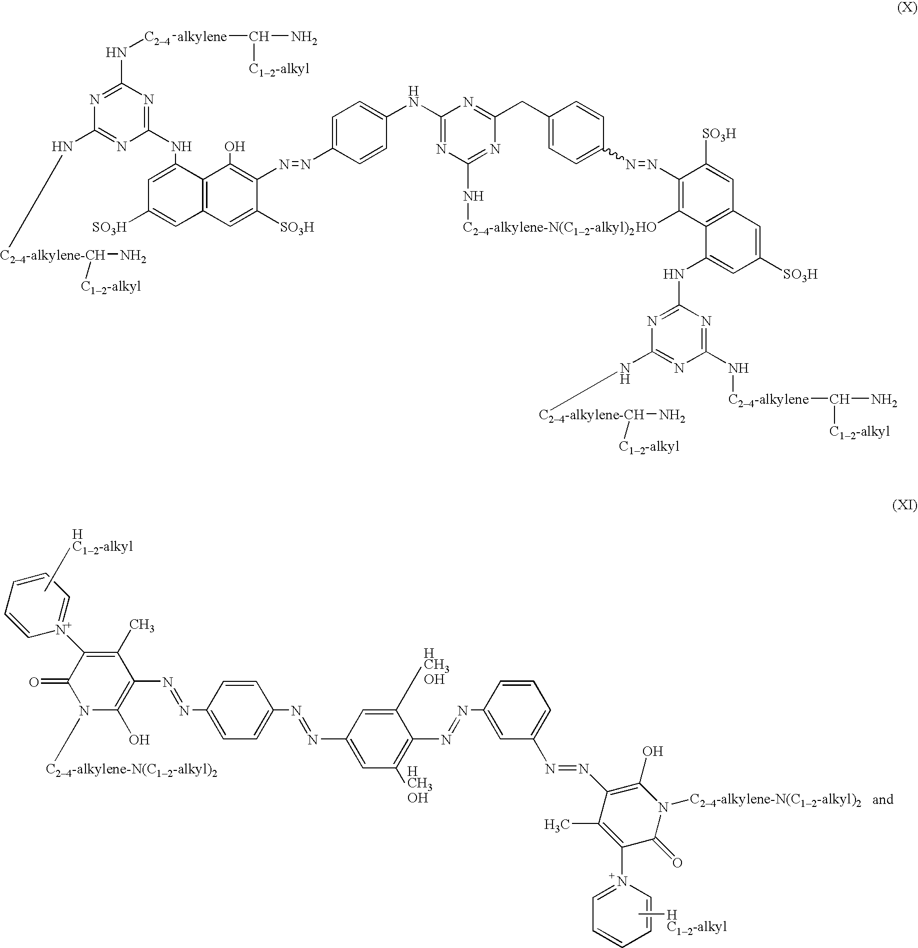

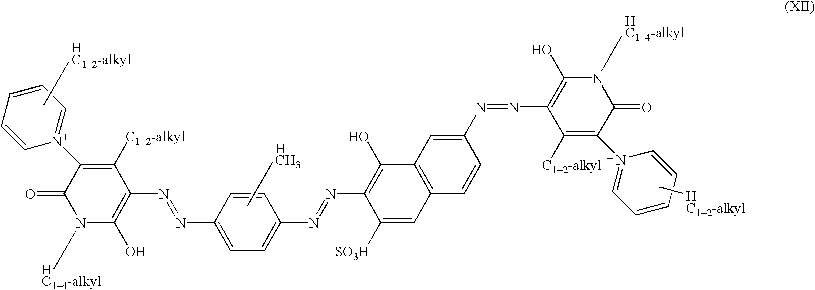

8. Composition for a use according to claims 1-7, comprising

| | |
|---|---|
| 0.01-10 parts | of a compound of the formula (I) and/or of the formula |
| | (II) and/or of the formula (Ill) and/or of the formula (IV) |
| 0.5-40 parts | of surface-active substances |
| 0-5 parts | of thickeners |
| 0.5-40 parts | of solubilising agents |
| 0-80 parts | of organic solvents |
| 5-50 parts | of water, |

the total of all parts always being 100.

9. Composition for a use according to claim 8, characterized in that gel-forming substances and/or antioxidants, penetration agents and/or sequestering agents and/or buffers and/or perfume oil and/or light protection agents and/or preservatives and/or hair-leansing preparations are additionally present.
